(19) United States
(12) Patent Application Publication
(10) Pub. No.: US 2010/0088108 A1
(43) Pub. Date: Apr. 8, 2010

(54) PHARMACEUTICAL RELATIONAL DATABASE

(75) Inventor: Paulo Machado, Mercerville, NJ (US)

Correspondence Address: Weiss & Arons, LLP, 1540 Route 202, Suite 8, Pomona, NY, 10970, US

(73) Assignee: AstraZeneca AB, Sodertalje (SE)

(21) Appl. No.: 12/247,264

(22) Filed: Oct. 8, 2008

(51) Int. Cl. G06Q 50/00; G06F 17/30

(52) U.S. Cl. 705/2; 707/E17.045

(57) ABSTRACT

Apparatus and methods for providing a pharmaceutical relational database are provided. A computer readable medium comprising a database according to the invention may include a first set of data relating to pharmacy eligibility for a medication. The database may also include a second set of data relating to health plan support for the medication, a third set of data relating to patient medication history; and a fourth set of data relating to special offers provided by a pharmaceutical company for the medication.

### FIELD OF TECHNOLOGY

Aspects of the disclosure relate to a database including pharmaceutical information.

### BACKGROUND

There are many drawbacks to the current pharmaceutical prescription system. These drawbacks include a relatively high incidence of Adverse Medication Events (ADEs) and error rates, miscommunication due to illegible handwriting, unclear abbreviations and dose designations, ambiguous orders and fax-related problems, complex prescription regimens and dosages, complex benefits plans and many others.

In order to deal with these problems associated with the current pharmaceutical prescription system, the National Institute of Medicine (IOM) recommended that all prescriptions be written and received electronically by the year 2010.

In order to comply with the recommendations of the IOM, industry has expended substantial resources in the area of e-prescribing. E-prescribing uses advanced technology to deliver medications to patients. E-prescribing has further been used to connect patients, physicians, pharmacists and payers within a secured network.

Nevertheless, the pharmaceutical companies that develop and market the pharmaceuticals have not played a part in the development of the national network of e-prescribing.

Accordingly, it would be desirable for the pharmaceutical companies to play a larger role in the development of e-prescribing in order to advance the various efficiencies that the pharmaceutical companies can bring to e-prescribing.

It would be desirable, as well, to provide a pharmaceutical relational database that includes information uniquely available to a pharmaceutical company that relates to a predetermined pharmaceutical.

### SUMMARY OF THE INVENTION

It is an object of the invention to provide a pharmaceutical relational database that includes information available to a pharmaceutical company that relates to a predetermined pharmaceutical.

A method, and apparatus, for providing a patient with a prescription for a medication according to the invention is provided. The method may include displaying on a graphical user interface a plurality of possible medications for prescription for the patient. This displaying of possible medications may occur on a page by page display—i.e., a single medication per page—or on a single display with multiple entries—i.e., multiple medications per page. The method may also include displaying the availability of a special offer related to one of the medications. The method may also include displaying the special offer information to one of a physician, a patient, or any other suitably situated individual.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 is a schematic diagram of apparatus that may be used in accordance with the principles of the invention;

FIG. 2 is an illustrative flow diagram according to the invention;

FIG. 3 is another illustrative flow diagram according to the invention;

FIG. 4 is yet another illustrative flow diagram according to the invention;

FIG. 5 is an information display according to the invention;

FIG. 6 is a conventional display of patient medication history data; and

FIG. 7 is a graphical user interface for use with the current invention.

### DETAILED DESCRIPTION OF THE INVENTION

In the following description of the various embodiments, reference is made to the accompanying drawings, which form a part hereof, and in which is shown by way of illustration various embodiments in which the invention may be practiced. It is to be understood that other embodiments may be utilized and structural and functional modifications may be made without departing from the scope and spirit of the present invention.

As will be appreciated by one of skill in the art upon reading the following disclosure, various aspects described herein may be embodied as a method, a data processing system, or a computer program product. Accordingly, those aspects may take the form of an entirely hardware embodiment, an entirely software embodiment or an embodiment combining software and hardware aspects.

Furthermore, such aspects may take the form of a computer program product stored by one or more computer-readable storage media having computer-readable program code, or instructions, embodied in or on the storage media. Any suitable computer readable storage media may be utilized, including hard disks, CD-ROMs, optical storage devices, magnetic storage devices, and/or any combination thereof. In addition, various signals representing data or events as described herein may be transferred between a source and a destination in the form of electromagnetic waves traveling through signal-conducting media such as metal wires, optical fibers, and/or wireless transmission media (e.g., air and/or space).

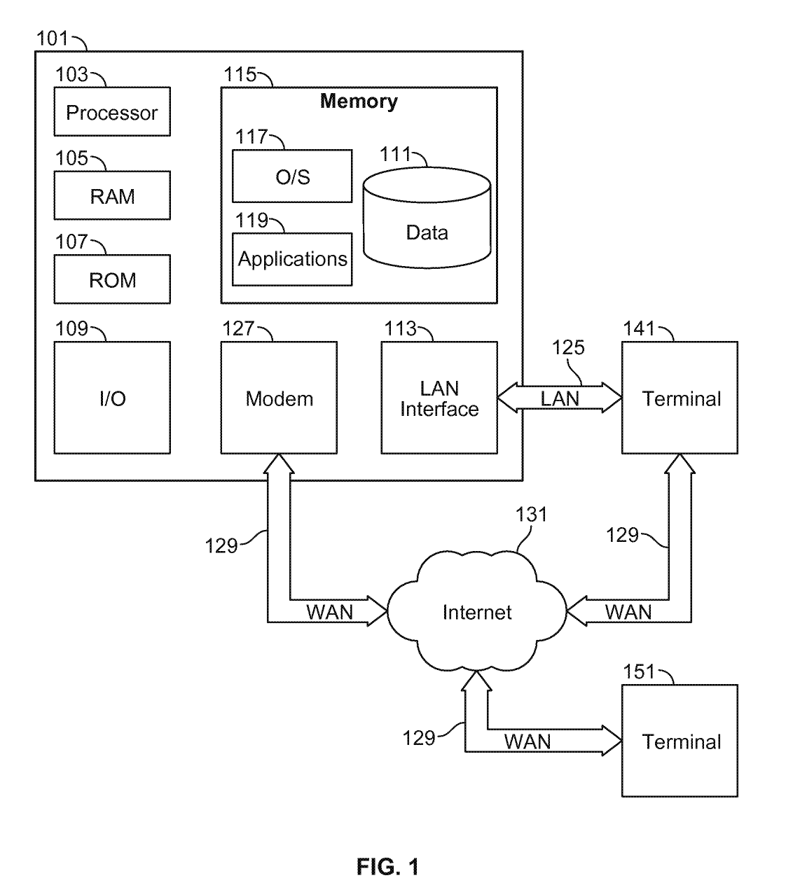

FIG. 1 is a block diagram that illustrates a generic computing device 101 (alternatively referred to herein as a “server”) that may be used according to an illustrative embodiment of the invention. The computer server 101 may have a processor 103 for controlling overall operation of the server and its associated components, including RAM 105, ROM 107, input/output module 109, and memory 125.

Input/output (“I/O”) module 109 may include a microphone, keypad, touch screen, and/or stylus through which a user of device 101 may provide input, and may also include one or more of a speaker for providing audio output and a video display device for providing textual, audiovisual and/or graphical output. Software may be stored within memory 125 and/or storage to provide instructions to processor 103 for enabling server 101 to perform various functions. For example, memory 125 may store software used by server 101, such as an operating system 117, application programs 119, and an associated database 121. Alternatively, some or all of server 202 computer executable instructions may be embodied in hardware or firmware (not shown). As described in detail below, database 121 may provide storage for patient demographic information, patient medical history information, formulary data and statistics, special offers information and any other suitable information.

Server 101 may operate in a networked environment supporting connections to one or more remote computers, such as terminals 141 and 151. Terminals 141 and 151 may be personal computers or servers that include many or all of the elements described above relative to server 101. The network connections depicted in FIG. 1 include a local area network (LAN) 125 and a wide area network (WAN) 129, but may also include other networks. When used in a LAN networking environment, computer 101 is connected to LAN 125 through a network interface or adapter 123. When used in a WAN networking environment, server 101 may include a modem 127 or other means for establishing communications over WAN 129, such as Internet 131. It will be appreciated that the network connections shown are illustrative and other means of establishing a communications link between the computers may be used. The existence of any of various well-known protocols such as TCP/IP, Ethernet, FTP, HTTP and the like is presumed, and the system can be operated in a client-server configuration to permit a user to retrieve web pages from a web-based server. Any of various conventional web browsers can be used to display and manipulate data on web pages.

Additionally, application program 119, which may be used by server 101, may include computer executable instructions for invoking user functionality related to communication, such as email, short message service (SMS), and voice input and speech recognition applications.

Computing device 101 and/or terminals 141 or 151 may also be mobile terminals including various other components, such as a battery, speaker, and antennas (not shown).

A physician may use a terminal such as 141 or 151 to utilize a pharmaceutical relational database according to the invention. The client may communicate with the intermediary using a transmission platform. Patient demographic information, medication history information, formulary ID and formulary lists, prescriber IDs and pharmacy IDs, may be stored in memory 125. Applications 119 may include a real-time e-prescribing application.

The invention is operational with numerous other general purpose or special purpose computing system environments or configurations. Examples of well known computing systems, environments, and/or configurations that may be suitable for use with the invention include, but are not limited to, personal computers, server computers, hand-held or laptop devices, mobile phones and/or other personal digital assistants (“PDAs”), multiprocessor systems, microprocessor-based systems, set top boxes, programmable consumer electronics, network PCs, minicomputers, mainframe computers, distributed computing environments that include any of the above systems or devices, and the like.

The invention may be described in the general context of computer-executable instructions, such as program modules, being executed by a computer. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

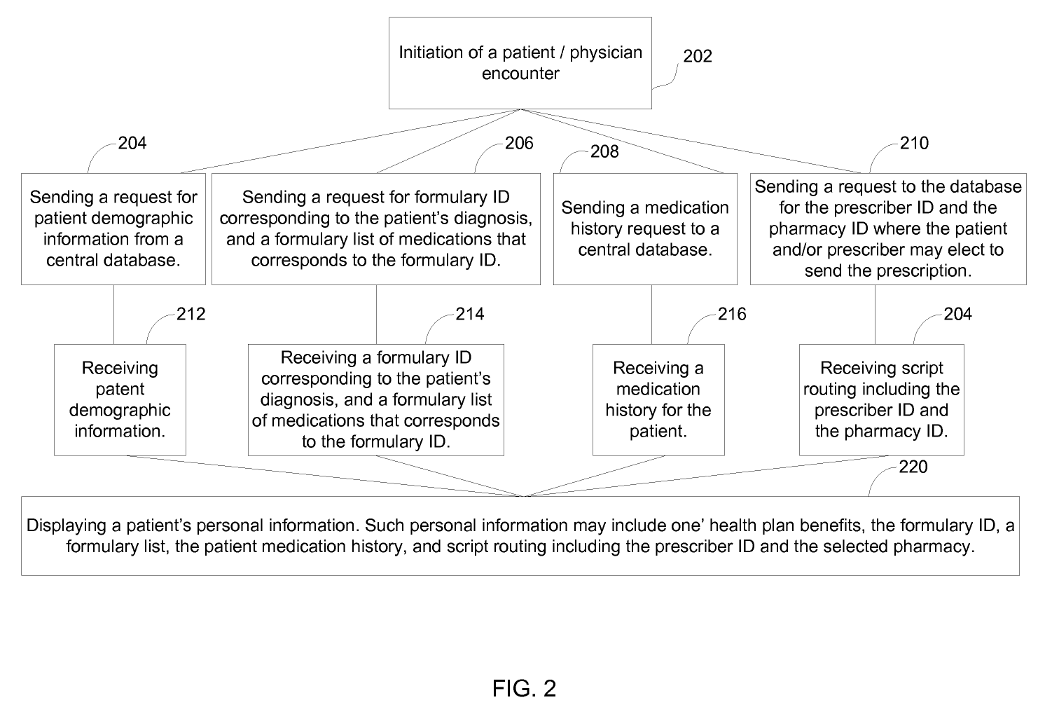

FIG. 2 shows an illustrative flow diagram according to the invention. Step 202 shows the initiation of a patient/physician encounter. Following (or, alternatively, prior to, or simultaneously to) initiation of the patient encounter, step 204 shows sending a request for patient demographic information from a central database. Step 206 shows sending a request for formulary ID for a type of medication that corresponds to the patient's diagnosis, and a formulary list of medications that correspond to the formulary ID.

Step 208 shows sending a medication history request to a central database. Step 210 shows sending a request to the database for the prescriber ID and the pharmacy ID where the patient and/or prescriber may elect to send the prescription.

Step 212 shows receiving patient demographic information. Step 214 shows receiving a formulary ID preferably corresponding to the patient's diagnosis, and a formulary list of medications that corresponds to the formulary ID. Step 216 shows receiving a medication history for the patient. Step 218 shows receiving script routing including the prescriber ID and the pharmacy ID.

Step 220 shows displaying a patient's personal information. Such personal information may include the patient's health plan benefits, the formulary ID, a formulary list, the patient medication history, and script routing including the prescriber ID and the selected pharmacy ID.

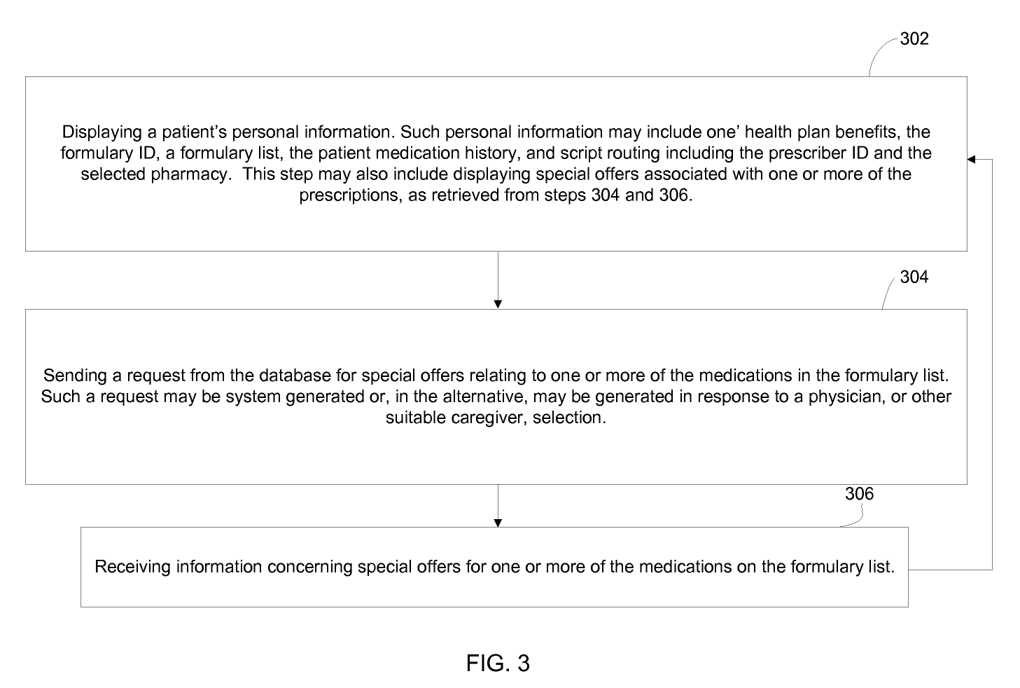

FIG. 3 shows an additional illustrative flow diagram that may form part of a process according to the invention. Step 302 shows displaying a patient's personal information, similar to step 220 shown in FIG. 2. Step 304 shows sending a request from the database for special offers relating to one or more of the medications in the formulary list. Such a request may be system generated or, in the alternative, may be generated in response to a physician, or other suitable caregiver, selection.

FIG. 3 shows another flow diagram according to the invention. Step 302 preferably includes displaying the information from step 220 shown in FIG. 2. In addition, step 302 may also include displaying information retrieved, as shown in step 304 and 306, concerning special offers.

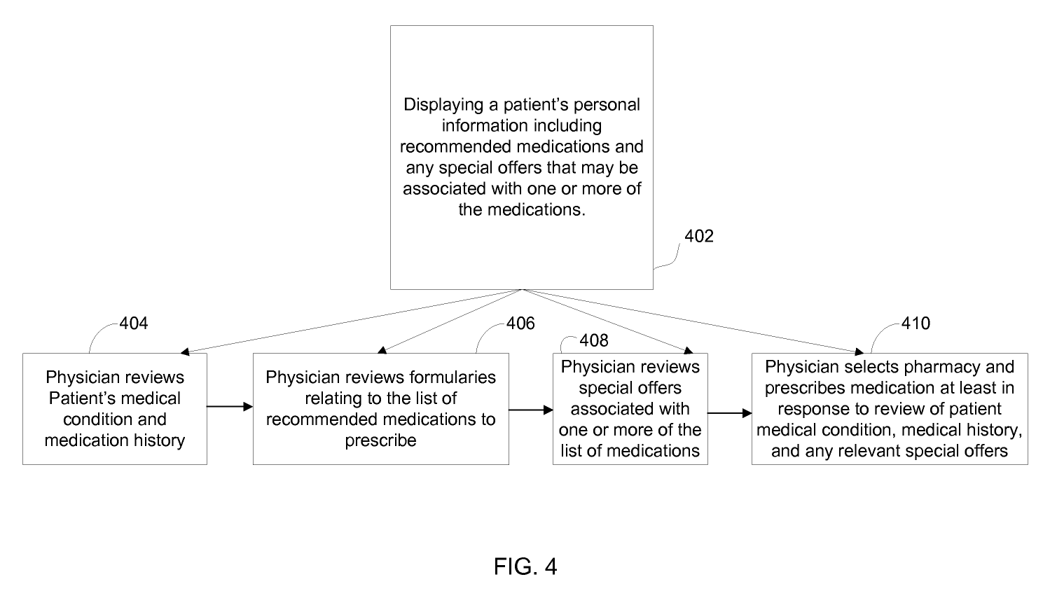

FIG. 4 shows displaying the information aggregated and displayed in step 302. FIG. 4 shows further the physician review of the patient medication history in step 404, review of the list of medications that may be prescribed in step 406, and review of any special offers associated with any of the prescribed medications as shown in step 408. Step 410 shows the physician selecting a pharmacy and prescribing the medication at least in response to review of patient medical condition, medical history, and any relevant special offers.

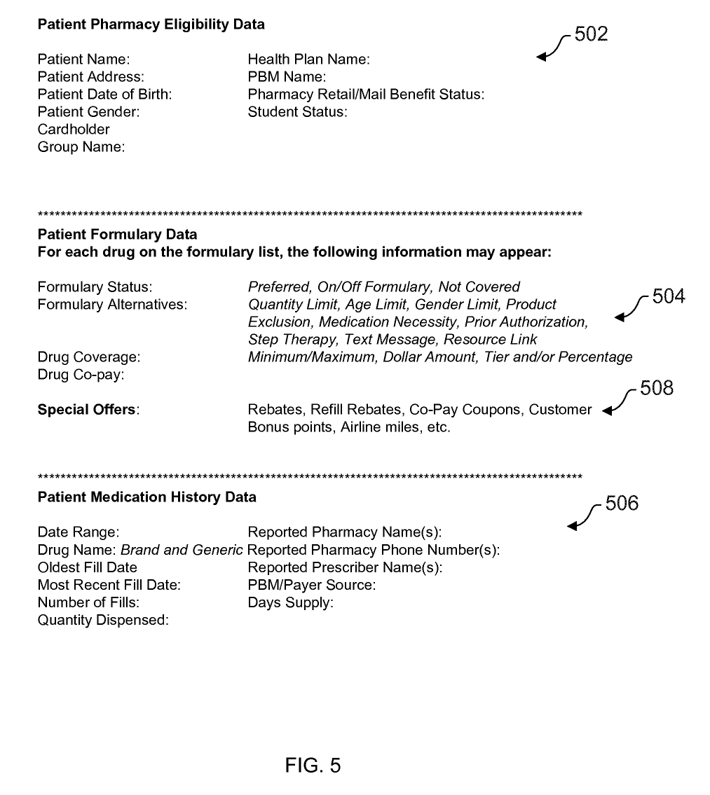

FIG. 5 shows an exemplary display of a display device, such as a PDA, that may form part of a system according to the invention. The display may include three sections: 1) patient pharmacy eligibility data 502 2) patient formulary data 504 and 3) patient medication history data 506.

Patient pharmacy eligibility data 502 may include: a patient name, a patient address, a patient date of birth, a patient gender, an insurance cardholder name, a group insurance name, a health plan name, a prescription benefit manager (“PBM”) name, a pharmacy retail/mail benefit status, and a patient student status.

Patient formulary data 504, which may be unique for each medication, may include health plan coverage of medications that may be prescribed to the patient, quantity limit of medications, age limit for users of the medications, gender restriction for users of the medications, medical necessity of the medications, prior authorization for the medications, and minimum and/or maximum health plan coverage of the medications.

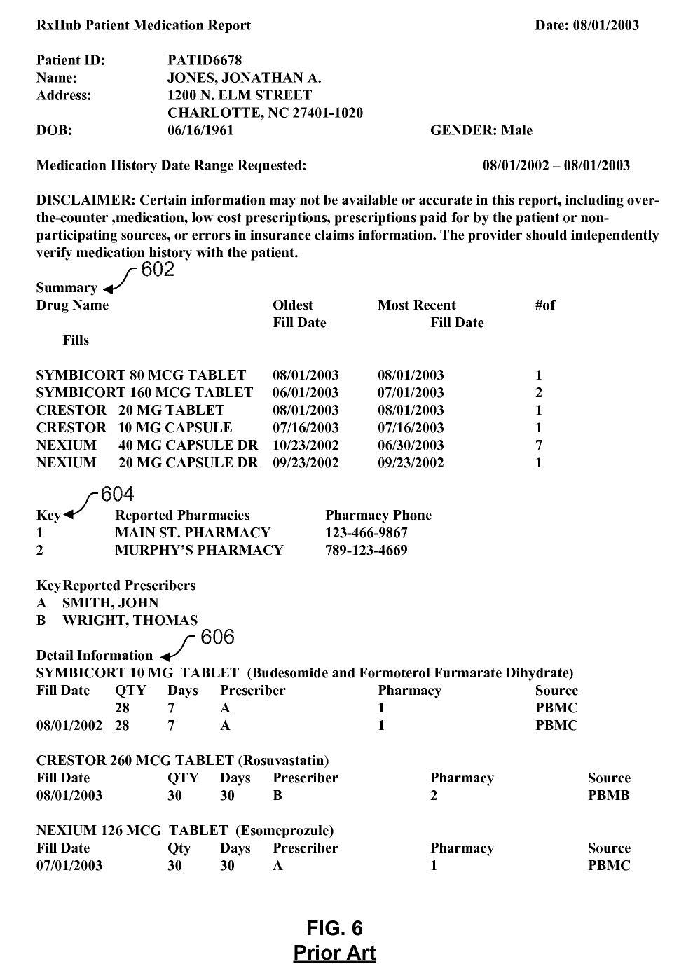

Patient medication history data 506 may include a predetermined date range, an oldest fill date, a most recent fill date, a number of fills, a reported pharmacy name, a reported pharmacy phone number, a reported prescriber name, a prescription benefits manager (“PBM”) source, days of supply, and quantity dispensed. A more detailed description of patient medical history is shown in FIG. 6.

Display 500 may further include special offers section 508. Special offers section may include offers related to one or more of the medications that are available to the patient. Such special offers may be defined for the purposes of this application to include a manufacturer's coupon for the medication, a co-pay rebate for the medication and/or a refills rebate for the medication, a medical device—e.g., in one embodiment of the invention a redeemable coupon for an item of value such as a glucometer, flowmeter, weight loss book, etc. may be provided—or any other suitable offer related to the medication.

FIG. 6 shows a conventional medical history display that may be used to illustrate a patient's medical history. It should be noted that a substantial portion of the display in FIG. 5 and the complete display shown in FIG. 6 appear in the RxHub Blueprint for e-prescribing which is incorporated by reference herein in its entirety and is available at the National Patient Health Information Network at www.rxhub.net.

The summary section 602 of the medication history shows the patient's medications sorted by the most recent fill date. In this illustration, when drugs are the same (with exception of strength and form), they may be listed together on the summary despite the date, with the most recent being first. If the date filled information is missing on the transaction, the drug can be shown at the top of the list and displayed without a date.

Key 604 lists the numbers and letters used in the detail information that correlate with pharmacies and health care professionals such as physicians. Pharmacies can be listed in alphabetical order and distinguished by their National Counsel for Prescription Drug Programs (“NCPDP”) ID's. The same pharmacy may be listed more than once if it has multiple locations.

Detailed Information 606 lists the Brand drug name first, then the (Generic drug name).

Fill Date: the date filled by the pharmacy.

Qty: the quantity of the medication dispensed.

Days: the number of days the medication will last.

Prescriber: the clinician prescribing the medication as found in the Key above.

Pharmacy: the pharmacy that filled the prescription as found in the Key above.

Source: the short name of the PBM that paid the claim.

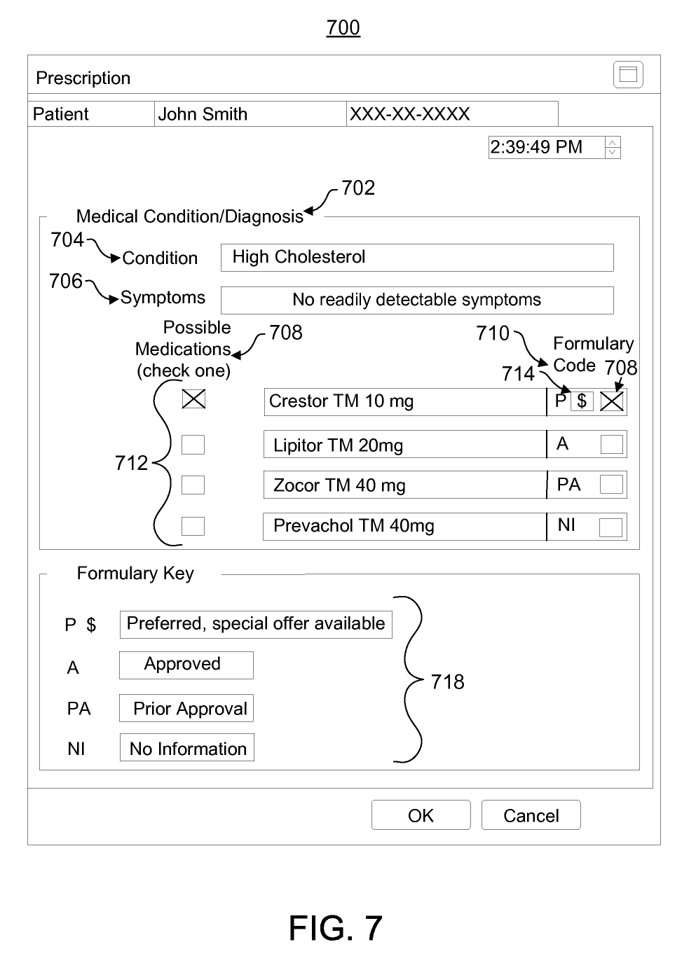

FIG. 7 shows one embodiment of a graphical user interface (“GUI”) 700 according to the invention. GUI 700 preferably includes a medical condition/diagnosis area 702.

Area 702 may include a condition field 704 and a symptoms field 706. Area 702 may also include a possible medications area 708 which may include all the medications listed at 712. Each of the medications may include a formulary code 710 which corresponds, at least in part, to the health plan coverage for the individual patient with respect to the medications.

The GUI according to the invention may also include a special offers symbol 714. Such a symbol may alert the physician or other suitable caregiver to the existence of a special offer concerning the listed medication. The physician can then either provide the patient with a coupon that the medication manufacturer gave to the physician. In some embodiments, the physician may be able to instruct the patient how the patient can take advantage of the special offer.

A special offers field 716 may be selectable according to the invention. Accordingly, when a physician selects the field, the special offer may be printed at the physician's office, at the patient's home, at a chosen pharmacy or at any other suitable workstation. Accordingly, the special offer can be generated at the time of prescription or at some other suitable time.

GUI may also include formulary key 718 which includes the various codes that the patient's help plan may have associated with the medication, as shown by code 710.

Aspects of the invention have been described in terms of illustrative embodiments thereof. A person having ordinary skill in the art will appreciate that numerous additional embodiments, modifications, and variations may exist that remain within the scope and spirit of the invention.

One of ordinary skill in the art will appreciate that the apparatus features described herein and illustrated in the Figures may be arranged in other than the recited configuration and that one or more of the features may be optional. Also, the methods described herein and illustrated in the Figures may be performed in other than the recited order and that one or more steps illustrated may be optional. The above-referenced embodiments may involve the use of other additional elements, steps, computer-executable instructions, or computer-readable data structures. In this regard, other embodiments are disclosed herein as well that can be partially or wholly implemented on a computer-readable medium, for example, by storing computer-executable instructions or modules or by utilizing computer-readable data structures.

Thus, systems and methods for providing a pharmaceutical relational database have been provided. Persons skilled in the art will appreciate that the present invention can be practiced by other than the described embodiments, which are presented for purposes of illustration rather than of limitation, and that the present invention is limited only by the claims that follow below.

What is claimed is:

1. A computer readable medium comprising a database, the database comprising:
a first set of data relating to pharmacy eligibility for a medication;
a second set of data relating to health plan support for the medication;
a third set of data relating to patient medication history; and
a fourth set of data relating to a special offer provided by a pharmaceutical company for the medication.

2. The computer readable medium of claim 1, the first set of data further comprising data that relates to at least one of a patient name, a patient address, a patient date of birth, a patient gender, an insurance cardholder name, a group insurance name, a health plan name, and a prescription benefit manager (“PBM”) name.

3. The computer readable medium of claim 1, the second set of data including at least one of health plan coverage of the medication, quantity limit of the medication, age limit for users of the medication, gender restriction for users of the medication, medical necessity of the medication, prior authorization for the medication, and minimum and/or maximum health plan coverage of the medication.

4. The computer readable medium of claim 1, the third set of data including least one of a predetermined date range, an oldest fill date, a most recent fill date, a number of fills, a reported pharmacy name, a reported pharmacy phone number, a reported prescriber name, a prescription benefit manager (“PBM”) source, days of supply, and quantity dispensed.

5. The computer readable medium of claim 1, the fourth set of data including data relating to a manufacturer's coupon for the medication.

6. The computer readable medium of claim 1, the fourth set of data including data relating a co-pay rebate for the medication.

7. The computer readable medium of claim 1, the fourth set of data including data relating to a refills rebate for the medication.

8. The computer readable medium of claim 1, wherein the first, second, third and fourth sets of data can be displayed on a computerized graphical device in the form of a multi-dimensional tabular or graphical display which show a correspondence between the data in the first, second, third and fourth sets of data.

9. The computer readable medium of claim 1, wherein the special offer comprises at least one of a manufacturer's coupon for the medication, a co-pay rebate for the medication, a refill rebate for the medication, a glucometer, flowmeter and a weight loss book.

10. A method of creating a relational database for use in informing a patient about a special offer provided by a pharmaceutical company, the method comprising:
creating a first set of data relating to health plan support for a medication;
creating a second set of data relating to patient medication history;
creating a third set of data relating to the special offer provided by a pharmaceutical company for the medication;
receiving a request to determine the acceptance parameters of a predetermined patient with respect to the medication, said acceptance parameters being based, at least in part on the first, second, and third sets of data; and
providing information regarding the special offer in response to the request.

11. The method of claim 10 further comprising creating a fourth set of data relating to pharmacy eligibility for the medication, the fourth set of data further comprising data that relates to at least one of a patient name, a patient address, a patient date of birth, a patient gender, an insurance cardholder name, a group insurance name, a health plan name, and a prescription benefit manager (“PBM”) name.

12. The method of claim 10, the first set of data including at least one of health plan coverage of the medication, quantity limit of the medication, age limit for users of the medication, gender restriction for users of the medication, medical necessity of the medication, prior authorization for the medication, and minimum and/or maximum health plan coverage of the medication.

13. The method of claim 10, the second set of data including least one of a predetermined date range, an oldest fill date, a most recent fill date, a number of fills, a reported pharmacy name, a reported pharmacy phone number, a reported prescriber name, a prescription benefit manager (“PBM”) source, days of supply, and quantity dispensed.

14. The method of claim 10, the third set of data including data relating to a manufacturer's coupon for the medication.

15. The method of claim 10, the third set of data including data relating a co-pay rebate for the medication.

16. The method of claim 10, the third set of data including data relating to a refills rebate for the medication.

17. The method of claim 10, further comprising displaying the first, second, and third sets of data on a computerized graphical device in the form of a multi-dimensional tabular or graphical display.

18. The method of claim 17 wherein the display visually indicates a correspondence between the data in the first, second, and third sets of data.

19. The method of the claim 17 wherein the special offer comprises at least one of a manufacturer's coupon for the medication, a co-pay rebate for the medication, a refill rebate for the medication, a glucometer, flowmeter and a weight loss book.

20. A method for providing a patient with a prescription for a medication, the method comprising:
displaying on a graphical user interface a plurality of possible medications for prescription for the patient; and
displaying the availability of a special offer related to one of the medications.

21. The method of claim 20 wherein the special offer is sponsored by the manufacturer of the medication.

22. The method of claim 20, wherein the displaying a plurality of possible medications for prescription for the patient is based on the patient health plan.

23. The method of claim 20, wherein the displaying a plurality of possible medications for prescription for the patient is based on patient medication history.

24. The method of claim 20, wherein the displaying a plurality of possible medications for prescription for the patient is based on the pharmacy eligibility for the plurality of medications.

25. The method of claim 20, wherein the special offer comprises at least one of a manufacturer's coupon for the medication, a co-pay rebate for the medication, a refill rebate for the medication, a glucometer, flowmeter and a weight loss book.

26. A method for providing information relating to medication being prescribed to a patient, the method comprising:
transmitting to a portable electronic device a plurality of possible medications for prescription for the patient; and
transmitting to the portable electronic device the availability of a special offer related to one of the medications.

27. The method of claim 26 wherein the special offer is sponsored by the manufacturer of the medication.

28. The method of claim 26, wherein the displaying a plurality of possible medications for prescription for the patient is based on the patient health plan.

29. The method of claim 26, wherein the displaying a plurality of possible medications for prescription for the patient is based on patient medication history.

30. The method of claim 26, wherein the displaying a plurality of possible medications for prescription for the patient is based on the pharmacy eligibility for the plurality of medications.

31. A portable electronic device configured to display information, the information relating to medication being prescribed to a patient, the portable electronic device comprising:
a display configured to display a plurality of possible medications for prescription for the patient and to display the availability of a special offer related to one of the medications.

32. The method of claim 31 wherein the special offer is sponsored by the manufacturer of the medication.

33. The method of claim 31, wherein the displaying a plurality of possible medications for prescription for the patient is based on the patient health plan.

34. The method of claim 31, wherein the displaying a plurality of possible medications for prescription for the patient is based on patient medication history.

35. The method of claim 31, wherein the displaying a plurality of possible medications for prescription for the patient is based on the pharmacy eligibility for the plurality of medications.

36. The method of claim 31, wherein the special offer comprises at least one of a manufacturer's coupon for the medication, a co-pay rebate for the medication, a refill rebate for the medication, a glucometer, a flowmeter and a weight loss book.
